(19) United States
(12) Patent Application Publication
(10) Pub. No.: US 2012/0107366 A1
(43) Pub. Date: May 3, 2012

(54) Block Copolymer Systems and Their Use in Medical Devices

(76) Inventor: Mbiya Kapiamba, Cromwell, CT (US)

(21) Appl. No.: 13/219,740

(22) Filed: Aug. 29, 2011

Related U.S. Application Data
(60) Provisional application No. 61/409,596, filed on Nov. 3, 2010.

(51) Int. Cl. A61K 9/00; C08G 63/42; A61P 1/00; A61K 38/20; A61K 38/21; C08G 63/08; A61K 39/00

(52) U.S. Cl. 424/400; 525/418; 525/462; 424/184.1; 424/85.2; 424/85.4; 424/85.5; 424/85.6; 424/85.7

(57) ABSTRACT

The present disclosure relates to block copolymers, methods for their production, and the use of these copolymers in medical devices. In embodiments, the block copolymers may be used in tissue reinforcement including, for example, in hernia repair. The copolymers possess at least one block that is hydrophilic and fast degrading, and at least one other block that is hydrophobic and slower to degrade.

### CROSS-REFERENCE TO RELATED APPLICATIONS

This application claims the benefit of, and priority to, U.S. Provisional Patent Application No. 61/409,596, filed on Nov. 3, 2010, the entire disclosure of which is incorporated by reference herein.

### BACKGROUND

The present disclosure is related to copolymers suitable for forming medical devices. In embodiments, copolymers of the present disclosure possess hydrophilic blocks, which are fast to degrade, in combination with hydrophobic blocks, which are slow to degrade. Devices made from these block copolymers may be utilized in various medical applications including, but not limited to, tissue reinforcement.

Bioresorbable polymers, including copolymers, have been used in the fabrication of devices for implantation in living tissue. Examples of medical applications of such polymers and copolymers include sutures, hemostatic aids, intraosseous implants, slow-release drug delivery systems, and tissue reinforcement devices, including meshes and films for hernia repair, and the like.

To be effective, these devices should be made from materials that meet biological and physical requirements. The materials should be, at least in part, bioresorbable, nontoxic, noncarcinogenic, nonantigenic, and should demonstrate favorable mechanical properties such as flexibility, suturability in some cases, and amenability to custom fabrication. For certain indications, for example hernia repair, the device should be strong enough to provide tissue support and, in some cases, should also permit tissue integration after implantation.

Improved materials for medical devices remain desirable, including materials that can provide both enhanced strength for tissue reinforcement applications, as well as tissue integration.

### SUMMARY

The present disclosure provides block copolymers suitable for forming medical devices. In embodiments, a block copolymer of the present disclosure includes at least one hydrophilic block including a polyether; and at least one hydrophobic block including a random copolymer of an unsubstituted lactone in combination with a substituted lactone. Medical devices which may be formed of the copolymers of the present disclosure include hernia patches, tissue scaffolds, burn dressings, sponges, augmentation devices, breast prostheses, orthopedic devices, pins, plates, clamps, screws, vascular implants, arterial grafts, clips, staples, tacks, sutures, nerve channels, and combinations thereof

In embodiments, a medical device of the present disclosure may be a hernia patch including a block copolymer including at least one hydrophilic block including an ether; and at least one hydrophobic block including a random copolymer of an unsubstituted lactone in combination with a substituted lactone.

In embodiments, the medical devices of the present disclosure may also include at least one bioactive agent.

### DETAILED DESCRIPTION

The present disclosure provides block copolymers suitable for forming medical devices. The block copolymers of the present disclosure include two components: a hydrophilic, fast degrading, hydrophilic “A” block, and a hydrophobic, slow degrading “B” block.

The “A” block or blocks are formed from at least one type of monomeric unit including an ether, in some cases a polyether. Suitable ethers and/or polyethers which may be used to form the A block include, but are not limited to, alkylene oxides, including ethylene oxide and propylene oxide; alkyl substituted ethylene oxides such as ethyl, propyl, and butyl substituted ethylene oxide; polyalkylene oxides such as polyethylene oxide (“PEO”), polypropylene oxide (“PPO”), polyethylene oxide-co-polypropylene oxide (“PEO-PPO”), co-polyethylene oxide block or random copolymers; alkylene glycols including ethylene glycol and polyethylene glycol (“PEG”); polytetramethylene ether glycol, combinations thereof, and the like. Other suitable monomers or polymers which may be utilized to form the A block include, for example, polytetrahydrofuran, combinations thereof, and the like.

In embodiments, a polyethylene glycol (“PEG”) may be utilized to form the A block. It may be desirable to utilize a PEG with a weight average molecular weight of from about 200 to about 10,000 Daltons, in embodiments from about 900 to about 5,000 Daltons. Suitable PEGs include those commercially available from a variety of sources under the designations PEG 200, PEG 400, PEG 600, PEG 900, PEG 1,500, PEG 3,350 and PEG 5,000.

As noted above, the block copolymers of the present disclosure also include at least one relatively hydrophobic, slower to degrade, “B” block. The B block may include one or more recurring monomeric units. In embodiments, the B block may include lactone monomers and/or polymers.

In embodiments, the B block may be prepared by the random copolymerization of an unsubstituted lactone with a substituted lactone. Suitable unsubstituted lactones which may be utilized in forming the B block include, but are not limited to, caprolactones, valerolactones, dioxanones, dioxepanones, trimethylene carbonates, propiolactones, butyrolactone, combinations thereof, and the like.

Suitable substituted lactones utilized to form the B block include, but are not limited to, substituted compounds based upon oxepan-2-ones, 7-alkyl-oxepan-2-ones, 1,3-dioxepan-2-ones, 1,5-dioxepan-2-ones, 1,4-dioxepan-2-ones, 1,3-dioxepan-4-ones, combinations thereof, and the like. These lactones may be further substituted, in embodiments, with aryl groups including pyridines, aminoaryl, hydroxyaryl, and/or nitroaryl groups. These aryl groups can be further substituted with alkyl groups, aryl groups, and/or alkoxy groups as long as they have one or more hydrogen bond donors or hydrogen bond acceptors. The substituted alkyl and/or aryl groups may include functional groups such as hydroxyl, amino, alkoxy, alkylamino, alkylthio, combinations thereof, and the like.

In embodiments, suitable substituted lactones utilized to form the B block include oxepanones such as 3-(4-hydroxybenzyl)oxepan-2-one, 3-(4-aminobenzyl)oxepan-2-one, 3-(pyridine-4-ylmethyl)oxepan-2-one, combinations thereof, and the like.

In embodiments, the aromatic substituent carrying the hydrogen bond donor or the hydrogen bond acceptor group can be linked to the lactone monomer ring by a heterocyclic group such as a triazole group prepared by a Click chemistry reaction between an azido lactone and the corresponding acetylenic derivative substituted with the aromatic substituent carrying the hydrogen bond donor or the hydrogen bond acceptor group.

Where the B block is formed from a combination of starting monomers, i.e., unsubstituted lactones and substituted lactones, the relative ratios of one monomer to any of the other monomers may vary from about 10/90 to about 90/10, in embodiments from about 20/80 to about 80/20, in embodiments from about 70/30 to about 30/70.

In embodiments, the A block and B block may be linked by ester bonds, produced by a reaction including at least one cyclic anhydride, or urethane bonds, produced by a reaction with a selected diisocyanate.

For ester bonds, as noted above, these bonds may be formed by combining the A block with the B block in the presence of a cyclic anhydride. For example, the A block may be reacted with an anhydride, in embodiments a cyclic anhydride, to introduce carboxy groups on the A block. These pendant carboxy groups may then react with the B block forming ester bonds between the A block and B block.

Examples of suitable cyclic anhydrides include phthalic anhydride, tetrahydrophthalic anhydride, naphtalenic dicarboxylic anhydride, hexahydrophthalic anhydride, 5-norbornene-2,3-dicarboxylic anhydride, norbornene-2,3-dicarboxylic anhydride, naphtalenic dicarboxylic anhydride, succinic anhydride, 2-octene-1-yl-succinic anhydride, 2-nonene-1-yl-succinic anhydride, 2-decene-1-yl-succinic anhydride, 2-undecene-1-yl-succinic anhydride 2-dodecene-1-yl-succinic anhydride, maleic anhydride, (methyl)succinic anhydride, glutaric anhydride, 4-methylphthalic anhydride, 4-methylhexahydrophthalic anhydride, 4-methyltetrahydrophthalic anhydride, glycolic anhydride, combinations thereof, and the like.

In embodiments, a stoichiometric amount of the cyclic anhydride may be reacted with the A block to introduce the pendant carboxy groups thereon.

In an exemplary synthesis, the A block is slowly melted, then treated with at least 2 molar equivalents of the cyclic anhydride, followed by the addition of a base such as pyridine. The reaction is continued at the designated temperature for a specified amount of time, before being diluted with a solvent such as THF, ethyl acetate, methylene chloride etc., and purified by precipitation with a non-solvent such as ether, petroleum ether, hexane, or other non polar solvents.

For urethane bonds, the A block may be endcapped with an isocyanate to produce an isocyanate-functional compound. Where both ends of the A block are endcapped with an isocyanate, a diisocyanate-functional compound is produced. Suitable isocyanates for endcapping the A block include aromatic, aliphatic and alicyclic isocyanates. Examples include, but are not limited to, aromatic diisocyanates such as 2,4-toluene diisocyanate, 2,6-toluene diisocyanate, 2,2′-diphenylmethane diisocyanate, 2,4′-diphenylmethane diisocyanate, 4,4′-diphenylmethane diisocyanate, diphenyldimethylmethane diisocyanate, dibenzyl diisocyanate, naphthylene diisocyanate, phenylene diisocyanate, xylylene diisocyanate, 4,4′-oxybis(phenyl isocyanate), 4,4′-methylenebis(phenyl isocyanate), or tetramethylxylylene diisocyanate; aliphatic diisocyanates such as tetramethylene diisocyanate, hexamethylene diisocyanate, dimethyl diisocyanate, lysine diisocyanate, 2-methylpentane-1,5-diisocyanate, 3-methylpentane-1,5-diisocyanate or 2,2,4-trimethylhexamethylene diisocyanate; and alicyclic diisocyanates such as isophorone diisocyanate, cyclohexane diisocyanate, hydrogenated xylylene diisocyanate, hydrogenated diphenylmethane diisocyanate, hydrogenated trimethylxylylene diisocyanate, 2,4,6-trimethyl 1,3-phenylene diisocyanate, or commercially available materials including those sold under the DESMODURS® name from Bayer Material Science.

Methods for endcapping the A block with an isocyanate are within the purview of those skilled in the art. For example, the A block may be combined with a suitable diisocyanate at a molar ratio of A block to isocyanate of from about 1:2 to about 1:6, in embodiments from about 1:3 to about 1:5, in other embodiments about 1:4, and heated to a suitable temperature of from about 55° C. to about 75° C., in embodiments from about 60° C. to about 70° C., in other embodiments about 65° C. It may be desirable to agitate the components utilizing means within the purview of those skilled in the art, including stirring, mixing, blending, sonication, combinations thereof, and the like.

In some embodiments, the endcapping reaction may occur under an inert atmosphere, for example, under nitrogen gas. Catalysts, including alkoxides, stannous octoate, dibutyltin dilaurate, 1,4-diazabicyclo[2.2.2]octane (DABCO), combinations thereof, and the like, may be utilized in some embodiments to increase the rate of the endcapping reaction.

It may be desirable, in embodiments, to utilize an excess of diisocyanate in carrying out the reaction. The use of an excess of diisocyanate may suppress the polymerization reaction, thereby permitting one to tailor the resulting molecular weight of the resulting isocyanate functionalized block. In some embodiments the resulting diisocyanate-functional block may then be purified by removal of excess diisocyanate reagent by hot extraction with petroleum ether.

The isocyanate group on the A block may then react with the B block, thereby forming a urethane linkage.

The block formation and number of recurring units in each block that make up the copolymers of the present disclosure may vary according to the intended use of the copolymers so formed. For example, diblocks, triblocks, multiple blocks, and the like may be formed. The block copolymers may be used alone or may be blended with other polymers to obtain the desired properties. In the formation of medical devices from the copolymers, such as films and similar structures for tissue reinforcement, it may be desirable to utilize an “ABA” or “BAB” triblock structure.

In addition, for certain applications, end-capping of the block copolymers may be desired. End-capping may be accomplished by conventional means, as for example, acetylation, alkylation, silylation and the like. The copolymers of the present disclosure may also be subjected to chain extension and/or grafting with monomeric, oligomeric or polymeric reactions with various reactants.

In embodiments, the polylactone block may also contain monomers with hydrogen bond donating groups such as aniline or phenol. In embodiments, suitable hydrogen bond donor groups include hydroxyphenol, aminophenol, thiophenol, benzamide, combinations thereof, and the like. In other embodiments, the polylactone block may incorporate hydrogen bond acceptor groups, such as pyridine groups, pyrimidine groups, pyrazines, combinations thereof, and the like. When the A block and B block are combined, a phase separation will occur where the polylactone of the B block will self-assemble and the PEG fragments of the A block will form another phase. The polylactone phase will have stronger affinity for itself due to the built-in hydrogen bonding between the monomers utilized to form the polylactone block. In embodiments, these two-phase systems can be processed to produce medical devices of the present disclosure, including films of controlled thickness for hernia repair.

The resulting block copolymer is biphasic, i.e., it possesses a fast degrading hydrophilic A block, and a slower degrading hydrophobic B block. In embodiments, the molecular weight of the individual blocks may be adjusted so that, upon processing, the copolymer may be strong and used for tissue repair, while still having altering rates of degradation so that a portion of the copolymer is rapidly degraded and eliminated from the body, creating space for tissue ingrowth in the remaining portion of the copolymer.

Suitable weight average molecular weights (Mw) of the A blocks may be from about 200 to about 10,000 Daltons, in embodiments from about 900 to about 5,000 Daltons, in embodiments from about 1,500 to about 3,500 Daltons. Suitable weight average molecular weights (Mw) of the B blocks may be from about 1,000 to about 150,000 Daltons, in embodiments from about 5,000 to about 100,000 Daltons, in embodiments from about 10,000 to about 50,000 Daltons.

Useful weight average molecular weights (Mw) of the copolymers for use in any particular situation will vary widely depending on the ultimate properties and characteristics it is desired to obtain for a medical device, such as modulus, strength, bioresorption and biodegradation rates, combinations thereof, and the like. In general, copolymer molecular weights useful for forming medical devices of the present disclosure, including films for hernia repair, are equal to or greater than about 10,000 Daltons. Suitable weight average molecular weights may be from about 10,000 to about 5,000,000 Daltons, in embodiments from about 20,000 to about 1,000,000 Daltons, in other embodiments from about 30,000 to about 500,000 Daltons.

In embodiments, the material may be a PEG-polylactone copolymer with controlled molecular weights of both blocks.

The copolymers of the present disclosure may be useful in the fabrication of totally or partially bioresorbable medical devices. The hydrophilic portion of the copolymer will possess a molecular weight such that degradation is fast and degradation products are easily eliminated from the body. Generally, once implanted in the body, the A block, in embodiments including a PEG, may thus degrade first, leaving a nanoporous scaffold formed of the hydrophilic B block, in embodiments including the polylactones described above. This scaffold may be conducive to cell infiltration and tissue integration.

For example, the A block may degrade in vivo over a time of from about 1 day to about 30 days after implantation, in embodiments from about 3 days to about 20 days after implantation, in embodiments from about 5 days to about 15 days after implantation. As noted above, the B block degrades at a slower rate. The B block may thus degrade in vivo over a time of from about 1 month to about 24 months after implantation, in embodiments from about 5 months to about 20 months after implantation, in embodiments from about 6 months to about 18 months after implantation.

The above copolymers may thus be suitable for use in tissue reinforcement applications, where the quicker degradation of the A block described above leaves behind the B block for continued reinforcement, while at the same time promoting tissue integration. In embodiments, the block copolymers of the present disclosure may be used to form films, foams, felts, meshes, and the like, which may be suitable for tissue reinforcement. In other embodiments, the copolymers of the present disclosure may be used to form filaments, which may be woven to form a mesh or other fabric or textile, or which may be used to form sutures. Illustrative of useful devices which may be fabricated from the copolymers of the present disclosure are hernia patches; tissue scaffolds; burn dressings; sponges; augmentation devices including breast prostheses; orthopedic devices such as pins, clamps, screws and plates; vascular implants or supports such as arterial grafts; clips; staples; tacks; nerve channels or supports; combinations thereof, and the like.

In embodiments, the copolymer of the present disclosure may be utilized to form a thin polymer film, which may be used to patch a defect, in embodiments a hernia, in vivo. The film may be utilized by itself or in conjunction with a fastening means including a suture, a screw, a tack, an adhesive, a sealant, combinations thereof, and the like. In embodiments, the film may be formed by curing by moisture in air, by heat, or other methods within the purview of those skilled in the art. The film may be cast as a thin film in which no bubbles are produced, to form a pore and defect free non-porous layer. In embodiments, the film has a thickness of from about 0.1 mm to about 2 mm, in other embodiments, from about 0.5 mm to about 1 mm.

The film may be used in a variety of applications including hernia repair, repairing fistulas, sealing anastomoses, as a buttress for suturing friable tissue, combinations thereof, and the like. The film provides strength and has elasticity to support the tissue.

It may be desirable to provide a variety of implants having different sizes so that a surgeon can select an implant of suitable size to treat a particular patient. This allows implants to be completely formed before delivery, ensuring that the smooth edge of the implant is properly formed under the control of the manufacturer. The surgeon would thus have a variety of differently sized (and/or shaped) implants to select the appropriate implant to use after assessment of the patient. In other embodiments, the patch can be cut to any desired size. The cutting may be carried out by a surgeon or nurse under sterile conditions such that the surgeon need not have many differently sized implants on hand, but can simply cut a patch to the desired size of the implant after assessment of the patient. In other words, the implant may be supplied in a large size and be capable of being cut to a smaller size, as desired.

Different shapes are suitable for repairing different defects in fascial tissue, and thus by providing a surgical implant which can be cut to a range of shapes, a wide range of defects in fascial tissue can be treated.

More broadly, the present disclosure recognizes that the implant can have any shape that conforms with an anatomical surface of a human or animal body that may be subject to a defect to be repaired by the implant.

For example, an anterior uterovaginal prolapse often is elliptical in shape or a truncated ellipse, whereas a posterior prolapse is circular or ovoid in shape. Accordingly, the implant shape may be any one of elliptical or truncated ellipse, round, circular, oval, ovoid or some similar shape to be used depending on the hernia or prolapse to be treated.

In this regard, while the surgical implant of the present disclosure may be useful for the repair of uterovaginal prolapse, it may also be used in a variety of surgical procedures including the repair of hernias.

In some embodiments, it may be desirable to secure the patch in place once it has been suitably located in the patient. The patch can be secured in any manner within the purview of those skilled in the art. Some examples include suturing the patch to strong lateral tissue, gluing the patch in place using a biocompatible glue, or using a surgical fastener, e.g., a tack, to hold the patch securely in place.

In embodiments it may be advantageous to use a biocompatible glue since it is fairly quick to apply glue to the area around the surgical implant. Additionally, the patch may include at least one capsule containing a biocompatible glue for securing the implant in place. In certain situations the patch may include up to about four capsules containing a biocompatible glue which may be provided around the perimeter of the surgical implant. The capsules may be hollow thin-walled spheres from about 3 mm to about 5 mm in diameter and may be made of gelatin.

Any biocompatible glue within the purview of one skilled in the art may be used. In embodiments useful glues include fibrin glues and cyanoacrylate glues.

In another embodiment, the patch of the present disclosure may be secured to tissue using a surgical fastener such as a surgical tack. Other surgical fasteners which may be used are within the purview of those skilled in the art, including staples, clips, helical fasteners, combinations thereof, and the like.

In embodiments, it may be advantageous to use surgical tacks as a surgical fastener to secure the patch to tissue. Tacks are known to resist larger removal forces compared with other fasteners. In addition, tacks only create one puncture, as compared to the multiple punctures created by staples. Tacks can also be used from only one side of the repair site, unlike staples, clips or other fasteners which require access to both sides of the repair site. This may be especially useful in the repair of a vaginal prolapse, where accessing the prolapse is difficult enough without having to access both sides of the prolapse. Suitable tacks which may be utilized to secure the patch of the present disclosure to tissue include, but are not limited to, the tacks described in U.S. Patent Application Publication No. 2004/0204723, the entire disclosure of which is incorporated by reference herein.

Suitable structures for other fasteners which may be utilized in conjunction with the patch of the present disclosure to secure same to tissue are within the purview of those skilled in the art and can include, for example, the suture anchor disclosed in U.S. Pat. No. 5,964,783, the entire disclosure of which is incorporated by reference herein. Additional fasteners which may be utilized and tools for their insertion include the helical fasteners disclosed in U.S. Pat. No. 6,562,051 and the screw fasteners disclosed in International Patent Application Publication No. WO 2004112841, the entire disclosures of each of which are incorporated by reference herein.

The surgical fasteners useful with the patch herein may be made from bioabsorbable materials, non-bioabsorbable materials, and combinations thereof. Suitable materials which may be utilized include those described in U.S. Patent Application Publication No. 2004/0204723 and International Patent Application Publication No. WO 2004112841, the entire disclosures of each of which are incorporated by reference herein. Examples of absorbable materials which may be utilized include trimethylene carbonate, caprolactone, dioxanone, glycolic acid, lactic acid, glycolide, lactide, homopolymers thereof, copolymers thereof, and combinations thereof. Examples of non-absorbable materials which may be utilized include stainless steel, titanium, nickel, chrome alloys, and other biocompatible implantable metals. In embodiments, a shape memory alloy may be utilized as a fastener. Suitable shape memory materials include nitinol.

Surgical fasteners utilized with the patch of the present disclosure may be made into any size or shape to enhance their use depending on the size, shape and type of tissue located at the repair site.

The surgical fasteners, e.g., tacks, may be used alone or in combination with other fastening methods described herein to secure the patch to the hernia, prolapse, or other repair site. For example, the patch may be tacked and glued, or sutured and tacked, into place.

The surgical fasteners may be attached to the patch in various ways. In embodiments, the ends of the patch may be directly attached to the fastener(s). In other embodiments, the patch may be curled around the fastener(s) prior to implantation. In yet another embodiment, the fastener may be placed inside the outer edge of the patch and implanted in a manner which pinches the patch up against the fastener and into the site of the injury.

Other polymeric components such as fillers and binders may be combined with the copolymers prior to and during the formation of films or other devices, or subsequent to their formation. These include, but are not limited to, polymers and copolymers including polyesters such as poly(butyleneterephthalate) and poly(ethyleneterephthalate); polyvinyl alcohol; polyvinylacetate and partially hydrolyzed forms thereof; hydrogel type polymers such as polyhydroxyethylmethacrylate, poly hydroxypropylmethacrylate, and the like; polysulfones such as polyphenylenesulfone; carbon; silicon carbide; halopolymers such as poly(tetrafluoroethylene) ethylene/tetrafluoroethylene copolymer; polydioxanone; polyglycolide-co-trimethylene carbonates; polylactides; poly-d-lactide; polylactide-co-caprolactone; poly-d,l-lactide; polycaprolactones; polyhydroxybutyrates; poly hydroxyvalerates; polyhydroxybutyrate-co-hydroxyvalerates; polyglycolide; polyurethanes; segmented polyurethanes; polyetherurethanes; polyurethane ureas; silicone rubber; and substances such as fibrin and its powder; natural or processed collagen; mono, di, tri, and polysaccharides; polyethylenes; polyamides; polypropylene; polycarbonates; poly(vinyl fluoride); poly(vinylidene fluoride); poly(vinyl butyral); cellulose such as, carboxylmethyl cellulose, cellulose acetate, ethylcellulose, and the like; ethylene-vinylacetate copolymers and hydrolyzed and partially hydrolyzed forms thereof; polyacrylonitrile; poly(vinylmethylether); and their derivative co-polymers; and the like.

Other components besides polymeric components may be combined with the copolymers before or as they are formed into devices of the present disclosure, or added to, coated onto, and the like, after their formation. These components include substances that will enhance certain of the desired properties of devices made from the copolymers of the present disclosure. Among the contemplated classes of such substances are plasticizers, lubricants, antioxidants, stabilizers of all kinds such as stabilizers for UV radiation, heat, moisture, and the like, as well as drugs for treatment of certain disorders or diseases. Materials such as calcium phosphate salts, ceramics, bioresorbable or otherwise, such as calcium hydroxyapatite, Bioglass, and calcium triphosphate may also be combined with the polymer. Components such as certain barium salts to render the devices formed from them radio-opaque are also within the contemplation of the present disclosure. Certain of these fillers, binders, additives and components can be removed or leached from such devices, at some stage, so that a porous or semi-porous devices, such as films, can be obtained. In addition, gas foaming during the formation of the device, either by gaseous, e.g., N₂, He, Ar, Ne, air, and the like, and/or their combinations, or chemical foaming agents, can be utilized to achieve a porous or somewhat porous structure.

In the embodiments, a device of the present disclosure, for example a hernia patch, may be coated with a bioresorbable coating to improve its patency. In embodiments, the desired coating may be an amorphous polycarbonate, which has some solubility in a solvent which is a non-solvent for the polymer forming the hernia patch. In general, the coating is applied to the patch by dissolving the coating polymer in a solvent which is a non-solvent for the patch, and then dipping the patch into the solution. Illustrative of useful solvents is dimethyl sulfoxide (DMSO), which will dissolve the materials which form the coating but not the copolymers which form the patch.

Thus, for those skilled in the art, it can be appreciated that aside from the polymeric composition and molecular weight and distribution of the copolymers of the present disclosure, processing particulars such as those described above can be profitably utilized or adjusted to achieve varying outcomes in biodegradation or bioresorption rates, hardness, toughness, softness, compliancy, adaptability, amenability to custom fabrication during manufacturing, and also in the field during the application of the device. This includes combining devices of the present disclosure, including films, with other films, fibers, fabrics, or devices.

In some embodiments, the copolymers of the present disclosure may be combined with one or more bioactive agents. The bioactive agent may be incorporated within the A block, incorporated within the B block, or both. Alternatively, the bioactive agent can be mixed with a copolymer including the A block and the B block prior to use. The term “bioactive agent”, as used herein, is used in its broadest sense and includes any substance or mixture of substances that may have clinical use. Consequently, bioactive agents may or may not have pharmacological activity per se, e.g., a dye. Alternatively, a bioactive agent could be any agent which provides a therapeutic or prophylactic effect, a compound that affects or participates in tissue growth, cell growth or cell differentiation, a compound that may be able to invoke a biological action such as an immune response, or could play any other role in one or more biological processes.

Examples of classes of bioactive agents which may be utilized in accordance with the present disclosure include antimicrobials, analgesics, antipyretics, anesthetics, antiepileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, gastrointestinal drugs, diuretics, steroids, lipids, lipopolysaccharides, polysaccharides, and enzymes. It is also intended that combinations of bioactive agents may be used in the present compositions.

Suitable antimicrobial agents which may be included as a bioactive agent in the compositions of the present disclosure include triclosan, also known as 2,4,4′-trichloro-2′-hydroxydiphenyl ether; chlorhexidine and its salts, including chlorhexidine acetate, chlorhexidine gluconate, chlorhexidine hydrochloride, and chlorhexidine sulfate; silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine; polymyxin; tetracycline; aminoglycosides such as tobramycin and gentamicin; rifampicin; bacitracin; neomycin; chloramphenicol; miconazole; quinolones such as oxolinic acid, norfloxacin, nalidixic acid, pefloxacin, enoxacin and ciprofloxacin; penicillins such as oxacillin and pipracil; nonoxynol 9; fusidic acid; cephalosporins; and combinations thereof. In addition, antimicrobial proteins and peptides such as bovine lactoferrin and lactoferricin B may be included as a bioactive agent in the compositions of the present disclosure.

Other bioactive agents which may be included as a bioactive agent in the compositions of the present disclosure include: local anesthetics; non-steroidal antifertility agents; parasympathomimetic agents; psychotherapeutic agents; tranquilizers; decongestants; sedative hypnotics; steroids; sulfonamides; sympathomimetic agents; vaccines; vitamins; antimalarials; anti-migraine agents; anti-parkinson agents such as L-dopa; anti-spasmodics; anticholinergic agents (e.g., oxybutynin); antitussives; bronchodilators; cardiovascular agents such as coronary vasodilators and nitroglycerin; alkaloids; analgesics; narcotics such as codeine, dihydrocodeinone, meperidine, morphine and the like; non-narcotics such as salicylates, aspirin, acetaminophen, d-propoxyphene and the like; opioid receptor antagonists, such as naltrexone and naloxone; anti-cancer agents; anti-convulsants; anti-emetics; antihistamines; anti-inflammatory agents such as hormonal agents, hydrocortisone, prednisolone, prednisone, non-hormonal agents, allopurinol, indomethacin, phenylbutazone and the like; prostaglandins and cytotoxic drugs; estrogens; antibacterials; antibiotics; anti-fungals; anti-virals; anticoagulants; anticonvulsants; antidepressants; antihistamines; and immunological agents.

Other examples of suitable bioactive agents which may be included in the composition of the present disclosure include viruses and cells; peptides; polypeptides and proteins, as well as analogs, muteins, and active fragments thereof; immunoglobulins; antibodies; cytokines (e.g., lymphokines, monokines, chemokines); blood clotting factors; hemopoietic factors; interleukins (IL-2, IL-3, IL-4, IL-6); interferons (β-IFN, α-IFN and γ-IFN); erythropoietin; nucleases; tumor necrosis factor; colony stimulating factors (e.g., GCSF, GM-CSF, MCSF); insulin; anti-tumor agents and tumor suppressors; blood proteins; gonadotropins (e.g., FSH, LH, CG, etc.); hormones and hormone analogs (e.g., growth hormone); vaccines (e.g., tumoral, bacterial and viral antigens); somatostatin; antigens; blood coagulation factors; growth factors (e.g., nerve growth factor, insulin-like growth factor); protein inhibitors; protein antagonists; and protein agonists; nucleic acids such as antisense molecules, DNA, and RNA; oligonucleotides; and ribozymes; naturally occurring polymers, including proteins such as collagen and derivatives of various naturally occurring polysaccharides such as glycosaminoglycans; peptide hydrolases such as elastase, cathepsin G, cathepsin E, cathepsin B, cathepsin H, cathepsin L, trypsin, pepsin, chymotrypsin, γ-glutamyltransferase (γ-GTP) and the like; sugar chain hydrolases such as phosphorylase, neuraminidase, dextranase, amylase, lysozyme, oligosaccharase and the like; oligonucleotide hydrolases such as alkaline phosphatase, endoribonuclease, endodeoxyribonuclease and the like. In some embodiments, where an enzyme is added, the enzyme may be included in a liposome or microsphere to control the rate of its release, thereby controlling the rate of degradation of the composition of the present disclosure. Methods for incorporating enzymes into liposomes and/or microspheres are within the purview of those skilled in the art.

A single bioactive agent may be utilized in the present compositions or, in alternate embodiments, any combination of bioactive agents may be utilized.

A variety of optional ingredients may also be added to the copolymers of the present disclosure. For example, a phospholipid surfactant that provides antibacterial stabilizing properties and helps dispense other materials in the compositions may be added to the compositions of the present disclosure. Imaging agents such as iodine or barium sulfate, or fluorine, can also be combined with the compositions of the present disclosure to allow visualization of the surgical area through the use of imaging equipment, including X-ray, MRI, and CAT scan.

It will be understood that various modifications may be made to the embodiments disclosed herein. Therefore, the above description should not be construed as limiting, but merely as an exemplification of preferred embodiments. Those skilled in the art will envision other modifications within the scope and spirit of the present disclosure. Such modifications and variations are intended to come within the scope of the following claims.

What is claimed is:

1. A block copolymer comprising:
at least one hydrophilic block comprising a polyether; and
at least one hydrophobic block comprising a random copolymer of an unsubstituted lactone in combination with a substituted lactone.

2. The block copolymer of claim 1, wherein the unsubstituted lactone and the substituted lactone of the hydrophobic block are joined by hydrogen bonding.

3. The block copolymer of claim 1, wherein the hydrophilic block is selected from the group consisting of polyethylene oxide, polypropylene oxide, polyethylene oxide-co-polypropylene oxide, alkyl substituted ethylene oxides, polyethylene glycol, polytetramethylene ether glycol, and combinations thereof

4. The block copolymer of claim 1, wherein the hydrophilic block comprises a polyethylene glycol having a weight average molecular weight of from about 200 to about 10,000 Daltons.

5. The block copolymer of claim 1, wherein the unsubstituted lactone is selected from the group consisting of caprolactones, valerolactones, dioxanones, dioxepanones, trimethylene carbonate, propiolactones, butyrolactone, and combinations thereof

6. The block copolymer of claim 1, wherein the substituted lactone is selected from the group consisting of oxepan-2-ones, 7-alkyl-oxepan-2-ones, 1,3-dioxepan-2-ones, 1,5-dioxepan-2-ones, 1,4-dioxepan-2-ones, 1,3-dioxepan-4-ones, trimethylene carbonates, valerolactones, and combinations thereof.

7. The block copolymer of claim 6, wherein the substituted lactone is substituted with a group selected from the group consisting of C1-C10 alkyl groups, aryl groups, substituted alkyl groups, and substituted aryl groups.

8. The block copolymer of claim 1, wherein the substituted lactone is substituted with a group selected from the group consisting of hydroxyphenol, aminophenol, thiophenol, benzamide, and combinations thereof.

9. The block copolymer of claim 1, wherein the substituted lactone is substituted with a group selected from the group consisting of pyridine, pyrimidine, pyrazine, and combinations thereof

10. The block copolymer of claim 1, wherein ratio of the unsubstituted lactone to the substituted lactone is from about 20/80 to about 80/20.

11. The block copolymer of claim 1, wherein the hydrophilic block has a weight average molecular weight of from about 200 to about 10,000 Daltons, and the hydrophobic block has a weight average molecular weight of from about 1,000 to about 150,000 Daltons.

12. The block copolymer of claim 1, wherein the hydrophilic block degrades in vivo over a time of from about 1 day to about 30 days after implantation, and the hydrophobic block degrades in vivo over a time of from about 1 month to about 24 months after implantation.

13. A medical device comprising the copolymer of claim 1.

14. The medical device of claim 13, wherein the medical device is selected from the group consisting of hernia patches, tissue scaffolds, burn dressings, sponges, augmentation devices, breast prostheses, orthopedic devices, pins, plates, clamps, screws, vascular implants, arterial grafts, clips, staples, tacks, sutures, nerve channels, and combinations thereof.

15. The medical device of claim 13, wherein the medical device further comprises at least one bioactive agent.

16. A hernia patch comprising a block copolymer comprising at least one hydrophilic block comprising an ether; and at least one hydrophobic block comprising a random copolymer of an unsubstituted lactone in combination with a substituted lactone.

17. The hernia patch of claim 16, wherein the unsubstituted lactone and the substituted lactone of the hydrophobic block are joined by hydrogen bonding.

18. The hernia patch of claim 16, wherein the hydrophilic block is selected from the group consisting of polyethylene oxide, polypropylene oxide, polyethylene oxide-co-polypropylene oxide, alkyl substituted ethylene oxides, polyethylene glycol, and combinations thereof.

19. The hernia patch of claim 16, wherein the hydrophilic block comprises a polyethylene glycol having a weight average molecular weight of from about 200 to about 10,000 Daltons.

20. The hernia patch of claim 16, wherein the unsubstituted lactone is selected from the group consisting of caprolactones, valerolactones, dioxanones, dioxepanones, trimethylene carbonate, propiolactones, butyrolactone, and combinations thereof

21. The hernia patch of claim 16, wherein the substituted lactone is selected from the group consisting of oxepan-2-ones, 7-alkyl-oxepan-2-ones, 1,3-dioxepan-2-ones, 1,5-dioxepan-2-ones, 1,4-dioxepan-2-ones, 1,3-dioxepan-4-ones, and combinations thereof.

22. The hernia patch of claim 21, wherein the substituted lactone is substituted with a group selected from the group consisting of C1-C10 alkyl groups, aryl groups, substituted alkyl groups, and substituted aryl groups.

23. The hernia patch of claim 16, wherein the substituted lactone is selected from the group consisting of 3-(4-hydroxybenzyl)oxepan-2-one, 3-(4-aminobenzyl)oxepan-2-one, 3-(pyridine-4-ylmethyl)oxepan-2-one, and combinations thereof.

24. The hernia patch of claim 16, wherein ratio of the unsubstituted lactone to the substituted lactone is from about 20/80 to about 80/20.

25. The hernia patch of claim 16, wherein the first block has a weight average molecular weight of from about 200 to about 10,000 Daltons, and the second block has a weight average molecular weight of from about 1,000 to about 150,000 Daltons.

26. The hernia patch of claim 16, wherein the hydrophilic block degrades in vivo over a time of from about 1 day to about 30 days after implantation, and the hydrophobic block degrades in vivo over a time of from about 1 month to about 24 months after implantation.

27. The hernia patch of claim 16, wherein the patch further comprises at least one bioactive agent.

28. The hernia patch of claim 16, wherein the patch comprises a film having a thickness of from about 0.1 mm to about 2 mm.
